(19) United States
(12) Patent Application Publication
(10) Pub. No.: US 2026/0225983 A1
(43) Pub. Date: Aug. 6, 2026

(54) HYDROFORMYLATION PROCESS

(71) Applicant: Dow Technology Investments LLC, Midland, MI (US)

(72) Inventor: Glenn A. Miller, South Charleston, WV (US)

(21) Appl. No.: 19/154,478

(22) PCT Filed: Mar. 4, 2024
(86) PCT No.: PCT/US24/18319

§ 371 Date: Aug. 7, 2025

Related U.S. Application Data
(60) Provisional application No. 63/500,021, filed on May 4, 2023.

(51) Int. Cl. C07C 45/50; C07C 45/80; C07C 45/82; C07C 45/86

(52) U.S. Cl. CPC C07C 45/50; C07C 45/80; C07C 45/82; C07C 45/86

(57) ABSTRACT

A hydroformylation process is disclosed having a reaction fluid comprising (a) at least one acidic compound selected from a phosphorus acidic compound or carboxylic acid compound, (b) a metal-organophosphorus ligand complex catalyst that comprises a metal of Group 8, 9 or 10 complexed with an organophosphorous ligand, and, optionally, (c) free organophosphorus ligand. This reaction fluid is contacted with an aqueous extraction fluid to facilitate the separation of at least some of acidic compounds from the reaction fluid via an extraction zone aqueous effluent stream. The process is characterized by at least a portion of the aqueous extraction fluid being comprised of recycled water from the hydroformylation system or a subsequent processing step in an aldehyde production process wherein the water stream to be recycled contains the product aldehyde and this water stream is subjected to at least one distillation process followed by at least one water/aldehyde phase separation process to produce the aqueous extraction fluid.

### BACKGROUND OF THE INVENTION

The invention relates to a hydroformylation process and further to a reduction in the amount of water needed for catalyst conditioning and reduced wastewater.

It is known that aldehydes can be produced by reacting an olefinically unsaturated compound with carbon monoxide and hydrogen in the presence of a rhodium-organophosphite ligand complex catalyst, and that preferred processes involve continuous hydroformylation and recycling of the catalyst solution as is disclosed, for example, in U.S. Pat. Nos. 4,148,830; 4,717,775 and 4,769,498. Such aldehydes have a wide range of known utility and are useful, for example, as intermediates for hydrogenation to aliphatic alcohols, for aldol condensation to produce plasticizers, and for oxidation to produce aliphatic acids.

Notwithstanding the benefits of such rhodium-organophosphorous ligand complex catalyzed liquid recycle hydroformylation processes, stabilization of the catalyst and organophosphorous ligand is a primary concern. Loss of catalyst or catalytic activity due to undesirable reactions of the highly expensive rhodium catalysts are detrimental to the production of the desired aldehyde. Degradation of the organophosphorous ligand employed during the hydroformylation process can lead to the formation of detrimental species, such as poisoning organophosphorous compounds, inhibitors, or acidic by-products, that can lower the catalytic activity of the rhodium catalyst. Production costs of the aldehyde product increase when productivity of the catalyst decreases.

Hydrolytic instability of hydrolyzable organophosphite ligands is a major cause of ligand degradation and catalyst deactivation for rhodium-organophosphorous ligand complex catalyzed hydroformylation processes. All organophosphites are susceptible to hydrolysis to some degree, the rate of hydrolysis generally being dependent on the stereochemical nature of the organophosphite. Typically, the bulkier the steric environment around the phosphorus atom, the slower the hydrolysis rate. For example, tertiary triorganophosphites, such as triphenylphosphite, are more susceptible to hydrolysis than diorganophosphites, such as those disclosed in U.S. Pat. No. 4,737,588, and organopolyphosphites such as those disclosed in U.S. Pat. Nos. 4,748,261 and 4,769,498. All such hydrolysis reactions invariably produce phosphorus acidic compounds that catalyze the hydrolysis reactions.

For example, the hydrolysis of a tertiary organophosphite produces a phosphonic acid diester, which is hydrolyzable to a phosphonic acid monoester, which in turn is hydrolyzable to H₃PO₃ (phosphorous acid). Moreover, hydrolysis of the ancillary products of side reactions, such as between a phosphonic acid diester and the aldehyde or between certain organophosphite ligands and an aldehyde, can lead to production of undesirable strong aldehyde acids, e.g., n-C₃H₇CH(OH)P(O)(OH)₂.

Even highly desirable sterically-hindered organobisphosphites that are not very hydrolyzable can react with the aldehyde product to form poisoning organophosphites, e.g., organomonophosphites, which are catalytic inhibitors and which are far more susceptible to hydrolysis and the formation of such aldehyde acid by-products, e.g., hydroxy alkyl phosphonic acids, as shown, for example, in U.S. Pat. Nos. 5,288,918 and 5,364,950. Further, the hydrolysis of organophosphite ligands may be considered to be autocatalytic in view of the production of such phosphorus acidic compounds, e.g., H₃PO₃, aldehyde acids, such as hydroxy alkyl phosphonic acids, H₃PO₄ and the like, and if left unchecked the catalyst system of a continuous liquid recycle hydroformylation process will become more and more acidic over time. The eventual build-up of an unacceptable amount of phosphorus acidic materials can cause the total destruction of the organophosphite present, thereby rendering the hydroformylation catalyst totally ineffective (deactivated) and the valuable rhodium metal susceptible to loss, e.g., due to precipitation and/or deposition on the walls of the reactor. For example, in U.S. Pat. No. 5,741,944, a buffered extractor can be used to remove acidic species as they are formed, but this extraction is done outside of the reactor system and can be overwhelmed in some cases. The acid mitigation does not occur under the high temperature and multiple hour residence time conditions of the reactor, thus some degradation may occur before the acid neutralization can occur. Also, sodium-based oxy-acid buffers have shown a tendency to deposit Na-based solids (primarily of neutralized acidic species) that can cause severe operating difficulties, including plant shutdowns.

Numerous methods have been proposed to maintain catalyst and/or organophosphite ligand stability. For instance, U.S. Pat. No. 5,288,918 suggests adding to the reaction zone a catalytic activity enhancing additive, such as water and/or a weakly acidic compound, U.S. Pat. No. 5,364,950 suggests adding to the reaction zone an epoxide to stabilize the organophosphite ligand-A further enhancement of the buffered extractor from U.S. Pat. No. 5,741,944 is taught in U.S. Pat. No. 8,884,072, wherein a water-washing step is added to remove metal salts derived from the oxyacid salt buffer prior to recycling the catalyst solution to the reaction zone.

Another means to remove the acidic contaminants is given in U.S. Pat. No. 5,763,677 which employs an ion exchange resin to remove the impurities and recycle the purified water back to the extraction zone. However, replacing the ion exchange resin is expensive and regenerating the resin produces a significant aqueous waste stream to be disposed of.

U.S. Pat. No. 5,744,649 and KR2019005328A teach a process to use unbuffered water to extract the acids. However, maintaining the desired effective pH of the catalyst solution requires a very large flow of de-ionized water, which results in elevated product, ligand and catalyst loss due to entrainment or dissolution in the water phase. U.S. Pat. Nos. 5,744,649 and 5,763,677 further teach distilling the initial aqueous extraction effluent such as in a “steam stripper” to recover the aldehyde dissolved in the initial aqueous effluent. This recovers valuable product and reduces the COD loading on the wastewater treatment facility. This process takes advantage of a butyraldehyde/water azeotrope. The process involves distilling a portion of the water then condensing and phase separating the organic layer (composed mostly of aldehyde product which is sent on for further processing) and an aqueous phase which is returned to the distillation system but ultimately is discarded in the distillation tails stream (i.e., not recycled to the hydroformylation process). Column 36 line 35 of U.S. Pat. No. 5,744,649 teaches that the water used in the extraction process should be free of organic matter.

CN111320532A teaches distilling the aqueous effluent from the catalyst fluid extraction zone to generate a distilled feed to use in the water washing step described in U.S. Pat. No. 8,884,072. In contrast to U.S. Pat. Nos. 5,744,649 and 5,763,677, the distilled stream is not phase separated prior to recycling to the extraction process thus a heterogeneous flow is used which makes control of the water content in the washing step difficult. This also recycles organic impurities back to the hydroformylation system rather than removing them in downstream processing such as taught in U.S. Pat. Nos. 5,744,649 and 5,763,677.

Prior art buffered extractors have been based on metal salts of oxyacids such as NaₓH_{y}PO₄. The buffer is typically preformed and fed at a concentration of >0.1 mmol/L to a countercurrent extractor where the acids are neutralized and removed under carefully controlled pH conditions. It was presumed in the prior art that the control of the pH in the aqueous buffer phase corresponds to an effective acidity control in the reaction zone. Adding amines to water at these concentrations without an oxyacid salt buffer present gives unacceptably high pH values and heavies formation in the reaction fluid. To have sufficient buffer capacity, high levels of amines such as pyridine, trialkylamines, and the like gave unacceptably high aqueous pH values (>9). U.S. Pat. No. 10,131,608 teaches the addition of low levels of weak, water-soluble amines with an aqueous extraction process to mitigate acids which avoids the high pH issue.

Notwithstanding the value of the teachings of the prior art, the search for alternative methods and an even better and more efficient means for stabilizing the rhodium catalyst and organophosphite ligand employed remains an ongoing activity. It would be desirable to have a process to reduce or eliminate highly acidic species in the hydroformylation reaction zone in order to minimize ligand degradation while reducing poisoning phosphite levels without the fouling observed with metal salt buffers.

The above processes preferably employ an aqueous extraction process at some stage in the removal of acidic impurities or their derivatives from the catalyst-containing hydroformylation process fluid. These result in an aqueous purge which must be dealt with in a downstream wastewater treatment process. The water used for these extraction processes require purification prior to use then is disposed of in the wastewater treatment facility. It would be desirable to reduce the amount of water needed to do these extractions, especially in regions which do not have abundant water supplies, and to reduce the amount of water that needs to be treated in the wastewater facility.

### SUMMARY OF THE INVENTION

It has been found that within the hydroformylation process and processing steps downstream of the hydroformylation process, distilled and decanted water streams are generated which are suitable to be used for these extraction processes. These distilled water streams are essentially free of the acids and acidic ligand degradation materials that are the cause of ligand degradation and heavies formation cited above. The distilled water will have the product aldehyde present (e.g., butyraldehyde) typically near or below the solubility limit (but still one phase) and traces of the corresponding alcohols (e.g., butanols) but since the recycled water will be used either to make a buffered solution to be sent to an aldehyde-containing stream or used to directly wash an aldehyde-containing stream, this aldehyde will have no adverse effect on the extraction process. This also recycles a small amount of aldehyde minimizing product losses without recycling significant organic impurities. These streams were generated under inert atmosphere thus do not require deaeration processing.

Examples of these distilled water streams include but are not limited to:
1. water distilled out during a product-catalyst vaporization step wherein the over-head stream is then distilled again to remove non-condensable lights (excess syngas, N₂). The overhead of such a process, when condensed, can generate a two-phase liquid stream wherein the bottom phase comprises mostly water. A recent example of this syngas removal process is shown in CN115417746.
2. During aldehyde isomer separation, the distillation overhead will comprise water and mostly the branched isomer. This mixture can phase separate upon cooling to form a bottom layer comprising mostly water. This was shown in CN217612991U (line 19).
3. Aldolization of aldehydes generates at least one mole of water per mole of aldehyde and this water is typically vaporized during the aldolization, preferably in at least one reactive distillation process such as in U.S. Pat. No. 5,434,313.
4. An alkaline catalyst purge from the aldolization process (see such as in U.S. Pat. No. 5,434,313) may also be used after being redistilled. This aqueous stream contains significant amounts of caustic thus must be distilled prior to reuse for the hydroformylation extraction process.
5. The aqueous extractor tails can be subjected to distillation and a portion of the condensed overhead stream is decanted water stream (as shown in U.S. Pat. No. 5,744,649) can be used.

The invention is such a process comprising: (1) conducting in a reaction zone a hydroformylation reaction employing a reaction fluid comprising (a) at least one acidic compound selected from a phosphorus acidic compound or carboxylic acid compound, (b) a metal-organophosphorus ligand complex catalyst that comprises a metal of Group 8, 9 or 10 complexed with an organophosphorous ligand, and, optionally, (c) free organophosphorus ligand; (2) contacting at least a portion of the reaction fluid with a water-based fluid to remove at least a portion of the acidic impurities wherein the water-based fluid preferably comprises an acid-neutralizing agent; (3) at least partially separating in an extraction zone at least one some of acidic compounds and preferably neutralized acidic compounds from the reaction fluid; and (4) removing the acidic compound and preferably neutralized acidic compound from the extraction zone via an extraction zone aqueous effluent stream;
wherein at least a portion of the aqueous extraction fluid used in step (2) is recycled water from the hydroformylation system or a subsequent processing step wherein the water contains the product aldehyde, and this recycled water stream is derived from at least one distillation followed by at least one water/aldehyde phase separation process.

Surprisingly, the process of the invention provides a way to control ligand degradation without increased heavies formation, and reduced water demand and lower wastewater treatment.

### DETAILED DESCRIPTION OF THE INVENTION

The disclosed process comprises contacting a water-based fluid with a hydroformylation reaction fluid. The reaction fluid comprises (1) at least one acidic compound selected from a phosphorus acidic compound or carboxylic acid compound, (2) a metal-organophosphorus ligand complex catalyst that comprises a metal complexed with an organophosphorous ligand, and, optionally, (3) free organophosphorus ligand. The reaction fluid can be generated in a hydroformylation reaction zone. An extraction zone advantageously is employed in conjunction with the reaction zone as part of the product recovery system in which at least a portion of the reaction fluid will be contacted with an aqueous extraction fluid. Use of an optional acid-neutralization agent can produce an extraction zone aqueous effluent stream with an acceptable pH range and provides the extraction zone with acceptable buffering capacity. The pH of the extraction zone aqueous effluent stream is controlled by extracting the acid-neutralization agent (if present) and the neutralized acidic compound from the organic phase of the extraction zone. The use of weak amines such as taught in U.S. Pat. No. 10,131,608 allows extraction and forming a buffered aqueous solution in situ.

The process is further characterized by having at least a portion of the aqueous extraction fluid used in the extraction zone comprise recycled water from the hydroformylation system or a subsequent processing step in an aldehyde production process. The water stream to be recycled will contain some of the product aldehyde and this water stream will be subjected to at least one distillation process followed by at least one water/aldehyde phase separation process to produce the recycled water to be used in the aqueous extraction fluid.

All references to the Periodic Table of the Elements and the various groups therein are to the version published in the CRC Handbook of Chemistry and Physics, 72nd Ed. (1991-1992) CRC Press, at page I-10.

Unless stated to the contrary, or implicit from the context, all parts and percentages are based on weight and all test methods are current as of the filing date of this application. For purposes of United States patent practice, the contents of any referenced patent, patent application or publication are incorporated by reference in their entirety (or its equivalent US version is so incorporated by reference) especially with respect to the disclosure of definitions (to the extent not inconsistent with any definitions specifically provided in this disclosure) and general knowledge in the art.

As used herein, “a,” “an,” “the,” “at least one,” and “one or more” are used interchangeably. The terms “comprises,” “includes,” and variations thereof do not have a limiting meaning where these terms appear in the description and claims. Thus, for example, an aqueous composition that includes particles of “a” hydrophobic polymer can be interpreted to mean that the composition includes particles of “one or more” hydrophobic polymers.

Also herein, the recitations of numerical ranges by endpoints include all numbers subsumed in that range (e.g., 1 to 5 includes 1, 1.5, 2, 2.75, 3, 3.80, 4, 5, etc.). For the purposes of the invention, it is to be understood, consistent with what one of ordinary skill in the art would understand, that a numerical range is intended to include and support all possible subranges that are included in that range. For example, the range from 1 to 100 is intended to convey from 1.01 to 100, from 1 to 99.99, from 1.01 to 99.99, from 40 to 60, from 1 to 55, etc. Also herein, the recitations of numerical ranges and/or numerical values, including such recitations in the claims, can be read to include the term “about.” In such instances the term “about” refers to numerical ranges and/or numerical values that are substantially the same as those recited herein.

As used herein, the term “ppmw” means part per million by weight.

For purposes of this invention, the term “hydrocarbon” is contemplated to include all permissible compounds having at least one hydrogen and one carbon atom. Such permissible compounds may also have one or more heteroatoms. In a broad aspect, the permissible hydrocarbons include acyclic (with or without heteroatoms) and cyclic, branched and unbranched, carbocyclic and heterocyclic, aromatic and nonaromatic organic compounds that can be substituted or unsubstituted.

As used herein, the term “substituted” is contemplated to include all permissible substituents of organic compounds unless otherwise indicated. In a broad aspect, the permissible substituents include acyclic and cyclic, branched and unbranched, carbocyclic and heterocyclic, aromatic and nonaromatic substituents of organic compounds. Illustrative substituents include, for example, alkyl, alkyloxy, aryl, aryloxy, hydroxyalkyl, aminoalkyl, in which the number of carbons can range from 1 to 20 or more, preferably from 1 to 12, as well as hydroxy, halo, and amino. The permissible substituents can be one or more and the same or different for appropriate organic compounds. This invention is not intended to be limited in any manner by the permissible substituents of organic compounds.

As used herein, the term “hydroformylation” is contemplated to include, but is not limited to, all hydroformylation processes that involve converting one or more substituted or unsubstituted olefinic compounds or a reaction mixture comprising one or more substituted or unsubstituted olefinic compounds to one or more substituted or unsubstituted aldehydes or a reaction mixture comprising one or more substituted or unsubstituted aldehydes. The aldehydes may be asymmetric or non-asymmetric.

The terms “reaction fluid,” “reaction medium” and “catalyst solution” are used interchangeably herein, and may include, but are not limited to, a mixture comprising: (a) a metal-organophosphorous ligand complex catalyst, (b) free organophosphorous ligand, (c) aldehyde product formed in the reaction, (d) unreacted reactants, (e) a solvent for said metal-organophosphorous ligand complex catalyst and said free organophosphorous ligand, and, optionally, (f) one or more phosphorus acidic compounds formed in the reaction (which may be homogeneous or heterogeneous, and these compounds include those adhered to process equipment surfaces). The reaction fluid can encompass, but is not limited to, (a) a fluid in a reaction zone, (b) a fluid stream on its way to a separation zone, (c) a fluid in a separation zone, (d) a recycle stream, (e) a fluid withdrawn from a reaction zone or separation zone, (f) a withdrawn fluid being treated with an aqueous solution, (g) a treated fluid returned to a reaction zone or separation zone, (h) a fluid in an external cooler, and (i) ligand decomposition products and their salts.

For the purposes of the invention, the term “heavies” means compounds that have a boiling point higher than that of the desired aldehyde product(s).

As used herein, the term “extractor” means any suitable vessel or container, e.g., any vessel suitable for use as a liquid/liquid extractor, that provides a suitable means for thorough contact between the reaction fluid and an aqueous solution. This can encompass a single counter-current extraction process, or a mixer system followed by a settler/decanter system.

For the purposes of the invention, the term “extraction zone” means an equipment system that comprises at least one extractor. An extraction zone can have multiple extractors arranged in parallel, series, or both.

The term “extraction zone aqueous effluent stream” refers to an effluent stream from the extraction zone that has, as its source, an aqueous phase that results following contact of the catalyst solution with an aqueous solution in an extraction zone.

For the purposes of the invention, the term “reaction zone” mean an equipment system that comprises at least one reactor, and that feeds at least a portion of the liquid effluent to a product-catalyst separation zone, which can comprise an extraction zone. The term “first reactor” refers to the first reactor in the reaction zone.

“Hydrolyzable phosphorous ligands” are trivalent phosphorous ligands that contain at least one P—Z bond wherein Z is oxygen, nitrogen, chlorine, fluorine or bromine.

Examples include, but are not limited to, phosphites, phosphino-phosphites, bisphosphites, phosphonites, bisphosphonites, phosphinites, phosphoramidites, phosphino-phosphoramidites, bisphosphoramidites, fluorophosphites, and the like. The ligands may include chelate structures and/or may contain multiple P-Z moieties such as polyphosphites, polyphosphoramidites, etc. and mixed P-Z moieties such as phosphite-phosphoramidites, flurophosphite-phosphites, and the like.

The term “complex” as used herein means a coordination compound formed by the union of one or more electronically rich molecules or atoms capable of independent existence with one or more electronically poor molecules or atoms, each of which is also capable of independent existence. For example, the organophosphorous ligands employable herein may possess one or more phosphorus donor atoms, each having one available or unshared pair of electrons that are each capable of forming a coordinate bond independently or possibly in concert (e.g., via chelation) with the metal. Carbon monoxide, which is also properly classified as a ligand, can also be present and coordinated to the metal. The ultimate composition of the complex catalyst may also contain an additional ligand, e.g., hydrogen or an anion satisfying the coordination sites or nuclear charge of the metal. Illustrative additional ligands include, for example, halogen (Cl, Br, I), alkyl, aryl, substituted aryl, acyl, CF₃, C₂ F₅, CN, (R)₂PO and RP(O)(OH)O (wherein each R is the same or different and is a substituted or unsubstituted hydrocarbon radical, e.g., the alkyl or aryl), acetate, acetylacetonate, SO₄, PF₄, PF₆, NO₂, NO₃, CH₃, CH₂═CHCH₂, CH₃CH—CHCH₂, C₆H₅CN, CH₃CN, NH₃, pyridine, (C₂H₅)₃N, mono-olefins, diolefins and triolefins, tetrahydrofuran, and the like. The complex species are preferably free of any additional organic ligand or anion that might poison the catalyst or have an undue adverse effect on catalyst performance. It is preferred in the metal-organophosphite ligand complex catalyzed hydroformylation reactions that the active catalysts be free of halogen and sulfur directly bonded to the metal, although such may not be absolutely necessary.

The number of available coordination sites on the transition metal is well known in the art and depends upon the particular transition metal selected. The catalytic species may comprise a complex catalyst mixture of monomeric, dimeric or higher nuclearity forms, which forms preferably are characterized by at least one organophosphorus-containing molecule complexed per one molecule of metal, for example, rhodium. For instance, it is considered that the catalytic species of the preferred catalyst employed in the hydroformylation reaction may be complexed with carbon monoxide and hydrogen in addition to one or more organophosphorous ligand(s).

It is recognized that the term “pH” is properly defined only for aqueous systems. When the term “effective pH” is used in this disclosure, it refers to the pH of an aqueous extraction of an organic phase to represent the amount of acidity/alkalinity present in that organic phase.

Buffers are mixtures of acids and bases. For the purposes of the invention, a buffer is an aqueous solution consisting of a mixture of a weak acid and its conjugate base or a weak base and its conjugate acid.

In preferred embodiments, an acid-neutralizing agent is used to convert the acidic compounds to neutralized salts that are water soluble thus more easily removed in the aqueous extraction process. These acid-neutralizing agents are typically metal salts of oxyacids such as described in U.S. Pat. No. 5,741,944 and weak amines such as described in U.S. Pat. No. 10,131,608 whose teachings are incorporated herein by reference. In some embodiments, the acid-neutralizing agent comprises at least one of triethanolamine, methyldiethanolamine, ethyldiethanolamine, tri(2-hydroxypropyl)amine, or ethoxylates of any of these. In other embodiments the acid neutralization agent comprises at least one anion selected from the group consisting of phosphate, carbonate, citrate, maleate, and borate compounds and at least one cation selected from the group consisting of ammonium and alkali metals and mixtures thereof.

If used, the acid neutralizing agent can advantageously be added to the aqueous extraction fluid as an aqueous solution, and such aqueous solution can be prepared using at least some of the recycled water.

The hydroformylation process, and conditions for its operation, are well known. Conducting a hydroformylation reaction involves contacting in a reaction zone CO, H₂, and at least one olefin in the presence of a hydroformylation catalyst under hydroformylation conditions sufficient to form at least one aldehyde product. The catalyst comprises as components a transition metal and a hydrolyzable organophosphorous ligand. Optional components for addition to the reaction zone include an epoxide and/or water.

Hydrogen and carbon monoxide may be obtained from any suitable source, including petroleum cracking and refinery operations. Syngas mixtures are a preferred source of hydrogen and CO.

Syngas (from synthesis gas) is the name given to a gas mixture that contains varying amounts of CO and H₂. Production methods are well known. Hydrogen and CO typically are the main components of syngas, but syngas may contain CO₂ and inert gases such as N₂ CH₄, and Ar. The ratio of H₂ to CO varies greatly but generally ranges from 1:100 to 100:1 and preferably between 1:10 and 10:1. Syngas is commercially available and is often used as a fuel source or as an intermediate for the production of other chemicals. The most preferred H₂:CO ratio for chemical production is between 3:1 and 1:3 and usually is targeted to be between about 1:2 and 2:1 for most hydroformylation applications.

The substituted or unsubstituted olefinic unsaturated reactants that may be employed in the hydroformylation process include both optically active (prochiral and chiral) and non-optically active (achiral) olefinic unsaturated compounds containing from 2 to 40, preferably 3 to 20, carbon atoms. These compounds are described in detail in US 2010/006980. Such olefinic unsaturated compounds can be terminally or internally unsaturated and be of straight-chain, branched chain or cyclic structures, as well as olefin mixtures, such as obtained from the oligomerization of propene, butene, isobutene, etc. (such as so called dimeric, trimeric or tetrameric propylene and the like, as disclosed, for example, in U.S. Pat. Nos. 4,518,809 and 4,528,403).

A solvent advantageously is employed in the hydroformylation process. Any suitable solvent that does not unduly interfere with the hydroformylation process can be used. By way of illustration, suitable solvents for rhodium catalyzed hydroformylation processes include those disclosed, for example, in U.S. Pat. Nos. 3,527,809; 4,148,830; 5,312,996; and 5,929,289. Non-limiting examples of suitable solvents include saturated hydrocarbons (alkanes), aromatic hydrocarbons, water, ethers, polyethers, aldehydes, ketones, nitriles, alcohols, esters, and aldehyde condensation products. Specific examples of solvents include: tetraglyme, pentanes, cyclohexane, heptanes, benzene, xylene, toluene, diethyl ether, tetrahydrofuran, butyraldehyde, and benzonitrile. The organic solvent may also contain dissolved water up to the saturation limit. Illustrative preferred solvents include ketones (e.g., acetone and methylethyl ketone), esters (e.g., ethyl acetate, di-2-ethylhexyl phthalate, 2,2,4-trimethyl-1,3-pentanediol monoisobutyrate), hydrocarbons (e.g., toluene), nitrohydrocarbons (e.g., nitrobenzene), ethers (e.g., tetrahydrofuran (THF)) and sulfolane. In rhodium catalyzed hydroformylation processes, it may be preferred to employ, as a primary solvent, aldehyde compounds corresponding to the aldehyde products desired to be produced and/or higher boiling aldehyde liquid condensation by-products, for example, as might be produced in situ during the hydroformylation process, as described for example in U.S. Pat. Nos. 4,148,380 and 4,247,486. The primary solvent will normally eventually additionally comprise both aldehyde products and heavies, due to the nature of the continuous process. The amount of solvent is not especially critical and need only be sufficient to provide the reaction medium with the desired amount of transition metal concentration. Typically, the amount of solvent ranges from about 5 percent to about 98 percent by weight, based on the total weight of the reaction fluid. Mixtures of solvents may be employed.

Also, it is to be understood that the preferred hydroformylation process of this invention, that is, the embodiment comprising preventing and/or lessening hydrolytic degradation of the organophosphite ligand and deactivation of the metal-organophosphite ligand complex catalyst by treating at least a portion of the hydroformylation reaction product fluid derived from the hydroformylation process and which also contains phosphorus acidic compounds formed during said hydroformylation process by introducing one or more acid neutralizing agents into said at least one reaction zone and/or said at least one separation zone sufficient to remove at least some amount of the phosphorus acidic compounds from said reaction product fluid, is also considered to be essentially a “non-aqueous” process. The primary solvent for the hydroformylation catalyst is an organic solvent such as described above and a separate water phase in the reaction zone is to be avoided. Some water will be dissolved in the organic fluid due to the solubility of water in the organic phase in the extractor but typically represents less than 5 wt % of the liquid phase in the reaction zone, preferably less than 2 wt %.

Thus, for example, a water-based fluid may be used to treat all or part of a reaction product fluid of a continuous liquid catalyst recycle hydroformylation process that has been removed from the reaction zone at any time prior to or after separation of the aldehyde product therefrom. More preferably said water-based treatment involves treating all or part of the reaction product fluid obtained after distillation of as much of the aldehyde product desired, for example, prior to or during the recycling of said reaction product fluid to the reaction zone.

For instance, a preferred mode would be to continuously pass all or part (for example, a slip stream) of the recycled reaction product fluid that is being recycled to the reaction zone through a liquid extractor containing the water-based fluid just before said catalyst containing residue is to re-enter the reaction zone.

Illustrative metal-organophosphorous ligand complexes employable in such hydroformylation reactions include metal-organophosphorous ligand complex catalysts. In preferred embodiments, the ligands are hydrolysable organophosphorous ligands. These catalysts, as well as methods for their preparation, are well known in the art and include those disclosed in the patents mentioned herein. In general, such catalysts may be preformed or formed in situ and comprise metal in complex combination with an organophosphorous ligand, carbon monoxide and optionally hydrogen. The ligand complex species may be present in mononuclear, dinuclear and/or higher nuclearity forms. However, the exact structure of the catalyst is not known.

The metal-organophosphorous ligand complex catalyst can be optically active or non-optically active. The metals can include Group 8, 9 and 10 metals selected from rhodium (Rh), cobalt (Co), iridium (Ir), ruthenium (Ru), iron (Fe), nickel (Ni), palladium (Pd), platinum (Pt), osmium (Os) and mixtures thereof, with the preferred metals being rhodium, cobalt, iridium and ruthenium, more preferably rhodium, cobalt and ruthenium, especially rhodium. Mixtures of these metals may be used. The permissible organophosphorous ligands that make up the metal-organophosphorous ligand complexes and free organophosphorous ligand include mono-, di-, tri- and higher polyorganophosphorus ligands. Mixtures of ligands may be employed in the metal-organophosphorous ligand complex catalyst and/or free ligand, and such mixtures may be the same or different.

The organophosphorous compounds that may serve as the ligand of the metal-organophosphorous ligand complex catalyst and/or free ligand may be of the achiral (optically inactive) or chiral (optically active) type and are well known in the art. Achiral organophosphorous ligands are preferred.

Among the organophosphorous ligands that may serve as the ligand of the metal-organophosphorous ligand complex catalyst are monoorganophosphite, diorganophosphite, triorganophosphite and organopolyphosphite compounds. Such organophosphorous ligands and methods for their preparation are well known in the art.

Representative monoorganophosphites may include those having the formula:

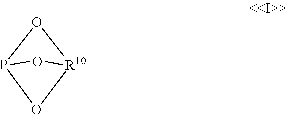

wherein R¹⁰ represents a substituted or unsubstituted trivalent hydrocarbon radical containing from 4 to 40 carbon atoms or greater, such as trivalent acyclic and trivalent cyclic radicals, e.g., trivalent alkylene radicals such as those derived from 1,2,2-trimethylolpropane and the like, or trivalent cycloalkylene radicals such as those derived from 1,3,5-trihydroxycyclohexane and the like. Such monoorganophosphites may be found described in greater detail, for example, in U.S. Pat. No. 4,567,306.

Representative diorganophosphites may include those having the formula:

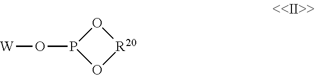

wherein R²⁰ represents a substituted or unsubstituted divalent hydrocarbon radical containing from 4 to 40 carbon atoms or greater and W represents a substituted or unsubstituted monovalent hydrocarbon radical containing from 1 to 18 carbon atoms or greater.

Representative substituted and unsubstituted monovalent hydrocarbon radicals represented by W in the above Formula (II) include alkyl and aryl radicals, while representative substituted and unsubstituted divalent hydrocarbon radicals represented by R²⁰ include divalent acyclic radicals and divalent aromatic radicals. Illustrative divalent acyclic radicals include, for example, alkylene, alkylene-oxy-alkylene, alkylene-S-alkylene, cycloalkylene radicals, and alkylene-NR²⁴-alkylene wherein R²⁴ is hydrogen or a substituted or unsubstituted monovalent hydrocarbon radical, e.g., an alkyl radical having 1 to 4 carbon atoms. The more preferred divalent acyclic radicals are the divalent alkylene radicals such as disclosed more fully, for example, in U.S. Pat. Nos. 3,415,906 and 4,567,302 and the like. Illustrative divalent aromatic radicals include, for example, arylene, bisarylene, arylene-alkylene, arylene-alkylene-arylene, arylene-oxy-arylene, arylene-NR²⁴-arylene wherein R²⁴ is as defined above, arylene-S-arylene, arylene-S-alkylene and the like. More preferably R²⁰ is a divalent aromatic radical such as disclosed more fully, for example, in U.S. Pat. Nos. 4,599,206, 4,717,775, 4,835,299, and the like.

Representative of a more preferred class of diorganophosphites are those of the formula:

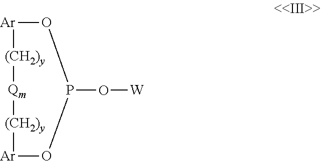

wherein W is as defined above, each Ar is the same or different and represents a substituted or unsubstituted aryl radical, each y is the same or different and is a value of 0 or 1, Q represents a divalent bridging group selected from —C(R³³)₂—, —O—, —S—, —NR²⁴—, Si(R³⁵)₂ and —CO—, wherein each R³³ is the same or different and represents hydrogen, an alkyl radical having from 1 to 12 carbon atoms, phenyl, tolyl, and anisyl, R²⁴ is as defined above, each R³⁵ is the same or different and represents hydrogen or a methyl radical, and m has a value of 0 or 1. Such diorganophosphites are described in greater detail, for example, in U.S. Pat. Nos. 4,599,206; 4,717,775; and 4,835,299.

Representative triorganophosphites may include those having the formula:

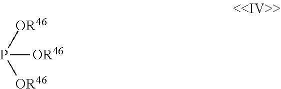

wherein each R⁴⁶ is the same or different and is a substituted or unsubstituted monovalent hydrocarbon radical e.g., an alkyl, cycloalkyl, aryl, alkaryl and aralkyl radicals that may contain from 1 to 24 carbon atoms. Illustrative triorganophosphites include, for example, trialkyl phosphites, dialkylaryl phosphites, alkyldiaryl phosphites, triaryl phosphites, and the like, such as, for example, trimethyl phosphite, triethyl phosphite, butyldiethyl phosphite, dimethylphenyl phosphite, triphenyl phosphite, trinaphthyl phosphite, bis(3,6,8-tri-t-butyl-2-naphthyl)methylphosphite, bis(3,6,8-tri-t-butyl-2-naphthyl)cyclohexylphosphite, tris(3,6-di-t-butyl-2-naphthyl)phosphite, bis(3,6,8-tri-t-butyl-2-naphthyl)phenylphosphite, and bis(3,6,8-tri-t-butyl-2-naphthyl)(4-sulfonylphenyl)phosphite, and the like. The most preferred triorganophosphite is triphenylphosphite. Such triorganophosphites are described in greater detail, for example, in U.S. Pat. Nos. 3,527,809 and 5,277,532.

Representative organopolyphosphites contain two or more tertiary (trivalent) phosphorus atoms and may include those having the formula:

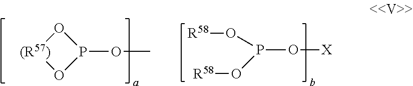

wherein X represents a substituted or unsubstituted n-valent organic bridging radical containing from 2 to 40 carbon atoms, each R⁵⁷ is the same or different and represents a divalent organic radical containing from 4 to 40 carbon atoms, each R⁵⁸ is the same or different and represents a substituted or unsubstituted monovalent hydrocarbon radical containing from 1 to 24 carbon atoms, a and b can be the same or different and each have a value of 0 to 6, with the proviso that the sum of a+b is 2 to 6 and n equals a+b. It is to be understood that when a has a value of 2 or more, each R⁵⁷ radical may be the same or different. Each R⁵⁸ radical may also be the same or different in any given compound.

Representative n-valent (preferably divalent) organic bridging radicals represented by X and representative divalent organic radicals represented by R⁵⁷ above, include both acyclic radicals and aromatic radicals, such as alkylene, alkylene-Qₘ-alkylene, cycloalkylene, arylene, bisarylene, arylene-alkylene, and arylene-(CH₂)_{y}-Qₘ-(CH₂)_{y}-arylene radicals, and the like, wherein each Q, y and m are as defined above in Formula (III). The more preferred acyclic radicals represented by X and R⁵⁷ above are divalent alkylene radicals, while the more preferred aromatic radicals represented by X and R⁵⁷ above are divalent arylene and bisarylene radicals, such as disclosed more fully, for example, in U.S. Pat. Nos. 4,769,498; 4,774,361: 4,885,401; 5,179,055; 5,113,022; 5,202,297; 5,235,113; 5,264,616 and 5,364,950, and 5,527,950. Representative preferred monovalent hydrocarbon radicals represented by each R⁵⁸ radical above include alkyl and aromatic radicals.

Illustrative preferred organopolyphosphites may include bisphosphites such as those of Formulas (VI) to (VIII) below:

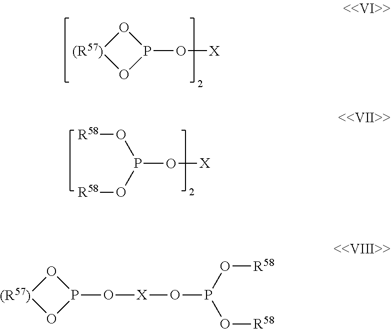

wherein each R⁵⁷, R⁵⁸ and X of Formulas (VI) to (VIII) are the same as defined above for Formula (V). Preferably each R⁵⁷ and X represents a divalent hydrocarbon radical selected from alkylene, arylene, arylene-alkylene-arylene, and bisarylene, while each R⁵⁸ radical represents a monovalent hydrocarbon radical selected from alkyl and aryl radicals. Organophosphite ligands of such Formulas (V) to (VIII) may be found disclosed, for example, in U.S. Pat. Nos. 4,668,651; 4,748,261; 4,769,498; 4,774,361; 4,885,401; 5,113,022; 5,179,055; 5,202,297; 5,235,113; 5,254,741; 5,264,616; 5,312,996; 5,364,950; and 5,391,801.

Specific illustrative examples of such organophosphite ligands include the following: 2-t-butyl-4-methoxyphenyl(3,3′-di-t-butyl-5,5′-dimethoxy-1,1′-biphenyl-2,2′-diyl)phosphite, methyl(3,3′-di-t-butyl-5,5′-dimethoxy-1,1′-biphenyl-2,2′-diyl)phosphite, 6,6′-∥3,3′-bis(1,1-dimethylethyl)-5,5′-dimethoxy-|1,1′-biphenyl]-2,2′-diyl|bis(oxy)|bis-dibenzo[d,f][1,3,2]dioxaphosphepin, 6,6′-[[3,3′,5,5′-tetrakis(1,1-dimethylethyl)-[1,1′-biphenyl]-2,2′-diyl]bis(oxy)]bis-dibenzo[d,f][1,3,2]-dioxaphosphepin, (2R,4R)-di[2,2′-(3,3′,5,5′-tetrakis-tert-butyl-1,1-biphenyl)]-2,4-pentyldiphosphite, (2R,4R)di[2,2′-(3,3′-di-tert-butyl-5,5′-dimethoxy-1,1′-biphenyl)]-2,4-pentyldi phosphite, 2-[2-[[4,8,-bis(1,1-dimethylethyl), 2,10-dimethoxydibenzo-[d,f][1,3,2]dioxophosphepin-6-yl]oxy]-3-(1,1-dimethylethyl)-5-methoxyphenyl]methyl]-4-methoxy, methylenedi-2,1-phenylene tetrakis[2,4-bis(1,1-dimethylethyl)phenyl]ester of phosphorous acid, and [1,1′-biphenyl]-2,2′-diyl tetrakis[2-(1,1-dimethylethyl)-4-methoxyphenyl]ester of phosphorous acid.

The metal-organophosphorous ligand complex catalysts may be in homogeneous or heterogeneous form. For instance, preformed rhodium hydrido-carbonyl-organophosphorous ligand catalysts may be prepared and introduced into a hydroformylation reaction mixture. More preferably, the rhodium-organophosphorous ligand complex catalysts can be derived from a rhodium catalyst precursor that may be introduced into the reaction medium for in situ formation of the active catalyst. For example, rhodium catalyst precursors such as rhodium dicarbonyl acetylacetonate, Rh₂O₃, Rh₄(CO)₁₂, Rh₆(CO)₁₆, Rh(NO₃)₃, and the like may be introduced into the reaction mixture along with the organophosphorous ligand for the in-situ formation of the active catalyst. In a preferred embodiment, rhodium dicarbonyl acetylacetonate is employed as a rhodium precursor and reacted in the presence of a solvent with the organophosphorous ligand to form a catalytic rhodium-organophosphorous ligand complex precursor that is introduced into the reactor along with excess (free) organophosphorous ligand for the in-situ formation of the active catalyst. In any event, it is sufficient that carbon monoxide, hydrogen and the organophosphorous ligand are all ligands that are capable of being complexed with the metal and that an active metal-organophosphorous ligand catalyst is present in the reaction mixture under the conditions used in the hydroformylation reaction. Carbonyl and organophosphorous ligands may be complexed to the rhodium either prior to, or in situ during, the hydroformylation process.

By way of illustration, a preferred catalyst precursor composition consists essentially of a solubilized rhodium carbonyl organophosphite ligand complex precursor, a solvent and, optionally, free organophosphite ligand. The preferred catalyst precursor composition can be prepared by forming a solution of rhodium dicarbonyl acetylacetonate, an organic solvent and a organophosphite ligand. The organophosphorous ligand readily replaces one of the carbonyl ligands of the rhodium acetylacetonate complex precursor as witnessed by the evolution of carbon monoxide gas.

Accordingly, the metal-organophosphorus ligand complex catalyst advantageously comprise the metal complexed with carbon monoxide and an organophosphorous ligand, said ligand being bonded (complexed) to the metal in a chelated and/or non-chelated fashion.

Mixtures of catalysts can be employed. The amount of metal-organophosphorous ligand complex catalyst present in the reaction fluid need only be that minimum amount necessary to provide the given metal concentration desired to be employed and that will furnish the basis for at least the catalytic amount of metal necessary to catalyze the particular hydroformylation process involved such as disclosed, for example, in the above-mentioned patents. In general, catalytic metal, e.g., rhodium, concentrations in the range of from 10 ppmw to 1000 ppmw, calculated as free metal in the reaction medium, should be sufficient for most processes, while it is generally preferred to employ from 10 to 500 ppmw of metal, and more preferably from 25 to 350 ppmw of metal.

In addition to the metal-organophosphorous ligand complex catalyst, free organophosphorous ligand (i.e., ligand that is not complexed with the metal) may also be present in the reaction medium. The free organophosphorous ligand may correspond to any of the above-defined organophosphorous ligands discussed above. It is preferred that the free organophosphorous ligand be the same as the organophosphorous ligand of the metal-organophosphorous ligand complex catalyst employed. However, such ligands need not be the same in any given process. The hydroformylation process of this invention may involve from 0.1 moles or less to 100 moles or higher of free organophosphorous ligand per mole of metal in the reaction medium. Preferably, the hydroformylation process is carried out in the presence of from 1 to 50 moles of organophosphorous ligand per mole of metal present in the reaction medium. More preferably, for organopolyphosphites, from 1.1 to 4 moles of organopolyphosphite ligand are employed per mole of metal. Said amounts of organophosphorous ligand are the sum of both the amount of organophosphorous ligand that is bound (complexed) to the metal present and the amount of free organophosphorous ligand present. If desired, additional organophosphorous ligand can be supplied to the reaction medium of the hydroformylation process at any time and in any suitable manner, e.g., to maintain a predetermined level of free ligand in the reaction medium.

In one embodiment, the rhodium catalyst may be impregnated onto any solid support, such as inorganic oxides, (i.e., alumina, silica, titania, or zirconia) carbon, membranes, thin films, or ion exchange resins, supported on, or intercalated inside the pores of, a zeolite, glass or clay, insoluble polymer support, or may also be dissolved in a liquid film coating the pores of said zeolite or glass.

Illustrative metal-organophosphorous ligand complex catalyzed hydroformylation processes that may experience hydrolytic degradation include those processes as described, for example, in U.S. Pat. Nos. 4,148,830; 4,593,127; 4,769,498; 4,717,775; 4,774,361; 4,885,401; 5,264,616; 5,288,918; 5,360,938; 5,364,950; 5,491,266 and 7,196,230. Species containing the P-Z moiety that will likely undergo hydrolytic degradation include organophosphonites, phosphoramidites, and fluorophosphonites such as described WO 2008/071508, WO 2005/042458, and U.S. Pat. Nos. 5,710,344, 6,265,620, 6,440,891, 7,009,068, 7,145,042, 7,586,010, 7,674,937, and 7,872,156. Accordingly, the hydroformylation processing techniques that are advantageously employed may correspond to any known processing techniques such as, for example, gas recycle, liquid recycle, and combinations thereof. Preferred hydroformylation processes are those involving catalyst liquid recycle.

In one embodiment of the invention, substantially no metal salt buffer is added to the process. In one embodiment of the invention, substantially no sodium-based oxy-acid buffer is added to the process.

In a preferred embodiment, the process of the invention employs an aqueous extraction step together with the addition of low levels of a water-soluble but relatively weak basic amine. One function of the amine is to neutralize acidic impurities. The neutralized acids are salts, e.g., ammonium salts. It is desirable to remove these salts to prevent their accumulation, which can lead to fouling and side reactions of the salts. The preferred route to remove the excess amine additive and neutralized acidic species is via an extractor in which a reaction fluid and an aqueous phase are brought together. In one embodiment of the invention, filtration, and ion exchange resins, such as taught in U.S. Pat. Nos. 7,495,134; 6,153,800; and 8,110,709, also can be used to remove at least a portion of the salts.

The amine advantageously may serve at least one of the following two functions: 1) it may neutralize acids, e.g. in the reaction zone, to mitigate ligand and catalyst degradation; and 2) it may control pH in the extraction step. The extraction step advantageously may serve at least one of the following three functions: 1) removing the neutralized acidic species (either as the salt or the acid) from the system, 2) providing water for poisoning phosphite degradation, and 3) removing excess amine to prevent amine buildup in order to avoid excessive heavies formation. The combination of the three features provides a self-balancing system where extremes of effective pH and heavies formation are avoided while still allowing controlled poisoning phosphite hydrolysis.

The amine may be added to the process at essentially any point so long as the desired concentration of amine is achieved. For example, the amine advantageously is added to the process in at least one of the reaction zones and/or the extraction zone. In one embodiment, the water-soluble amine is added to the process in more than one location. In one embodiment of the invention, the amine is added to the water feed to the extraction zone. In one embodiment of the invention, the amine is added to the first reactor. The water-soluble amine can be the same or different at the two addition points.

In one embodiment of the invention, the amine is primarily or entirely added to the reaction zone, and the rate of adding the water-soluble amine to the reaction fluid in the reaction zone is varied to control the pH of the extraction zone aqueous effluent stream in order to control the acidity of the reaction zone. In another embodiment of the invention, the amine is primarily added to the extraction zone, and the rate of addition of the water-soluble amine to the extraction zone is varied to control the pH of the extraction zone aqueous effluent stream. In one embodiment of the invention, the amine is introduced to the extraction zone as part of the aqueous feed stream. An amine/ammonium buffer is formed in situ as acid is delivered to the extraction zone via the organic phase, e.g., the reaction fluid from the reaction zone.

In a preferred embodiment, the water soluble, weak amine is added as a water solution rather than the pure amine. Such amines are typically viscous liquids or solids thus it is more convenient to add the amine as a dilute aqueous solution which also contributes to a more even distribution of the amine in the hydroformylation solution and easier process control. Likewise, the oxy-acid salts (such as sodium phosphates) are also preferably delivered to the extraction zone as water solutions. The water used for these solutions preferably comprise at least some recycled water as taught herein.

In a preferred embodiment, the acid neutralization agent advantageously is removed from the process with the water phase that exits the extraction zone. Thus, additional acid neutralization agent should be added to the process to maintain the desired concentration of the acid neutralization agent. The amount of the acid neutralization agent to add can be determined by observing the pH in the aqueous extraction zone, such as by measuring the pH of the aqueous stream leaving the extraction zone, e.g. the extractor tails stream. For example, the amount of weakly basic amine being added is sufficient to maintain the pH of this extraction zone aqueous effluent stream at from 4.5 to 9.0, preferably from 5.6 to 8.0, more preferably from 6.0 to 7.5, and most preferably, from 6.3 to 7.2. Occasionally, relatively higher pH values between 7.0 and 9.0 may be employed for short periods to mitigate high ligand decomposition periods, such as during a process upset when high ligand hydrolysis is observed, but this will result in a slow buildup of poisoning phosphite if continued for prolonged periods. Alternatively, relatively lower pH values (4.5 to 6.0) may be used for short times for maximum reactivity and olefin conversion (due to minimum poisoning phosphite concentration) at the cost of higher ligand usage. This situation may be present with lower quality feed or feeds containing high levels of secondary or internal olefins that require higher reactivity catalysts to maintain production rates. This scenario would not likely be economical for long periods due to ligand degradation costs, but the ability to return to the preferred pH range rapidly simply by increasing the amine addition rate shows the flexibility of the invention. Since the acid neutralization agent is removed by the extractor, raising and lowering the extractor pH is easily controlled by changing the acid neutralization agent addition rate to the process to affect this mitigation procedure without disturbing the hydroformylation production. pH values above 9 should be avoided due to low catalyst activity (from high poisoning phosphite levels) and excessive heavies formation. With oxy-acid salt buffers, the amount of buffered water flow and/or the ratio of salts (which determine the buffered water feed pH) and/or the molarity of the aqueous solution can be altered in a likewise manner to control the extractor tails pH.

Measurement of pH can be done using any means known to those skilled in the art including, for example, by conventional titration or commercially available pH meters with proper calibration. For the purposes of the invention, it is assumed that the organic phase acidity or “effective pH” of the organic phase correlates with the observed pH of an aqueous extraction such as the extractor tails.

In the preferred embodiment of the invention, at least a portion of the water-soluble amine is removed with the aqueous layer or phase of the extraction zone and, therefore, the amine does not build up in the organic phase. Since the water-soluble amine prefers to be in the aqueous phase, it is continuously removed and does not build up in the organic layer or phase. One step of the process of the invention involves at least partially separating in an extraction zone at least one neutralized acidic compound from the reaction fluid to form an extraction zone aqueous effluent stream and a treated hydroformylation reaction fluid. The separation involves contacting reaction fluid with an aqueous solution in the extraction zone, in which an extraction occurs. The contacting in the extraction zone not only removes free acidic compounds from the metal-organophosphorous ligand complex catalyst-containing reaction fluid, but it also removes the neutralized acidic compounds. Likewise, other acids are similarly removed such as carboxylic acids formed by the oxidation of the product aldehyde or heavies hydrolysis. The treated reaction fluid can preferably be returned to the reaction zone. The majority of the polar amine additive is removed into the water phase as the free amine or the ammonium salt in solution.

The aqueous solution fed to the extraction zone advantageously comprises a mixture of incoming water and recycled water. As used herein, the phrase “incoming water” refers to water sourced outside of the hydroformylation process and typically originates from rivers, wells, or seawater that has been treated to remove salt. The incoming water is usually de-ionized or distilled water and de-aerated such as described in U.S. Pat. No. 5,744,649 (column 36, lines 11-48). While not desirable, the incoming water feed may contain trace impurities, and/or additives or preservatives, e.g., anticorrosion additives, so long as they do not interfere with the hydroformylation catalyst. Some of these additives may have some intrinsic buffering effect, but in one embodiment of the invention they will contribute to less than 10% of the total acid neutralization performed in the extractor. As mentioned hereinabove, in one embodiment of the invention, all or part of the acid neutralization agent can be added to the aqueous solution feed to the extraction zone.

The phrase “recycled water” as used herein is water recovered from the hydroformylation system or a subsequent processing step wherein the water contains the product aldehyde, and this water stream is derived from at least one distillation followed by at least one water/aldehyde separation process. The recycled water used in the present invention has been distilled then decanted from an aldehyde-containing organic phase.

These processes are preferably done under inert atmosphere to avoid oxidation of the contained aldehyde thus do not require subsequent deaeration processing needed for the incoming water stream. The recycled water will contain substantial amounts of the product aldehyde (up to the solubility limit of the aldehyde in water at the temperature of the decantation). Other organic compounds may be present such as traces of aldehyde ether (e.g., dibutylether), alcohols, and possibly traces of butyric acid. The levels of these organic compounds can readily be determined by gas chromatography. In general, these compositions are well known and vapor-liquid equilibrium (VLE) modelling such as with ProII or ASPEN software packages can be used.

Butyric acids can be formed by the unintended oxidation of the aldehyde (e.g., air ingress) or hydrolysis of heavies. Its level can be reduced if a small amount of caustic is present in the distillation step since it will convert the acid to the non-volatile salt. In a preferred embodiment, the distillation step includes a portion of the aldolization catalyst purge as one of the feeds to the distillation as this also delivers caustic to the still bottoms. Butyric acid typically distills with the product due to an azeotrope (butyric acid/water: BP 100° C.; isobutyric acid/water: BP 99.3° C.) thus does not accumulate in the hydroformylation system but the equipment used to recycle the water should be compatible with traces of butyric acid to avoid corrosion issues. The buffered extraction process also removes butyric acid.

In one preferred embodiment, the organic effluent from the extraction zone is further treated with a water solution to further reduce dissolved impurities such as taught in U.S. Pat. No. 8,884,072 wherein a portion of the water used in this washing process is derived from recycled water as taught herein.

Recycled water can be used in the primary extraction process and/or in the washing process. In most cases, the incoming water can be the dominant source of water for the extraction process. In cases where an aldol system is present, the recycled water from the aldol unit can be sufficient to constitute all the water needed for the extraction process. In either case, the use of recycled water can represent a substantial reduction in the amount of water needed to operate the facility and a significant reduction in the wastewater being sent to the wastewater treatment facility.

The manner in which the acid neutralizing agent-containing reaction fluid from the reaction zone and the water feed are contacted in the extraction zone and/or the washing process described in U.S. Pat. No. 8,884,072, as well as the amount of aqueous solution, temperature, pressure and contact time, are not narrowly critical and need only be sufficient to obtain the results desired. A decrease in one of such conditions may be compensated for by an increase in one or more of the other conditions, while the corollary is also true. In general, liquid temperatures ranging from 10° C. to 120° C., preferably from 20° C. to 80° C., and more preferably from 25° C. to 60° C., should be suitable for most instances, although lower or higher temperatures may be employed if desired. Advantageously, the contacting in the extraction zone and/or washing process is carried out at a pressure ranging from ambient pressure to a pressure substantially higher than the reactor pressure, and the contact time may vary from a matter of seconds or minutes to a few hours or more. In general, it is preferred to pass the reaction fluid through the aqueous solution in an extractor column in a countercurrent fashion. The column can employ sieve trays, reciprocating-plates, structured or unstructured packing, and the like.

The extraction zone aqueous effluent stream advantageously is removed from the process and can be disposed of or used according to methods known to those skilled in the art. In a preferred embodiment, this effluent stream is sent to a distillation system and an overhead water/aldehyde stream is generated which is condensed to form two phases and the aqueous phase is recycled as taught herein. Of course, not all of the water can be recycled from this stream since the impurities (and any excess acid neutralization agent) removed has to be removed from the system via an aqueous purge.

Success in removing acidic compounds from the reaction fluid may be determined by measuring the rate of degradation (consumption) of the organophosphorous ligand present in the hydroformylation reaction medium. The consumption rate can vary over a wide range, e.g., from less than 0.06 up to 5 grams per liter per day and will be governed by the desired compromise between cost of ligand and treatment frequency to keep hydrolysis below autocatalytic levels. Preferably, the aqueous extraction is carried out in such a manner that the consumption of the desired organophosphorous ligand present in the hydroformylation reaction medium is maintained at an acceptable rate, e.g., less than 0.5 grams of ligand per liter per day, and more preferably less than 0.1 grams of ligand per liter per day, and most preferably less than 0.06 grams of ligand per liter per day. As the neutralization and extraction of acidic compounds into the aqueous solution of the extraction zone proceeds, the pH of the aqueous phase exiting the extraction zone will slowly decrease and the feed rate of water-soluble amine to the reaction zone can be increased to compensate.

As discussed above, the acidic compounds of concern are selected from phosphorus acidic compounds and/or carboxylic acid compounds. The phosphorus acidic compounds are primarily derived from degradation (hydrolysis) of hydrolysable organophosphorous ligands preferably used in the hydroformylation process. Many of these phosphorous acids are strong acids (effective pKa well below 3) and are of the most concern. The carboxylic acids may come from a variety of sources such as inadvertent oxidation of the product aldehyde or hydrolysis of ester heavies for example. Both materials are the most commonly found acidic materials in a hydroformylation process as described herein thus their mitigation are a focus of the invention. As used herein, the terms “acidic compound” and “acid impurities” will refer to either one or both of these sets of compounds. The term “neutralized acidic compound” will refer to the reaction product of an acid neutralizing agent with the acidic compound to form a salt (e.g., an ammonium salt of the phosphorous acid compound).

The removal of at least some amount of phosphorus acidic compounds, for example, H₃PO₃, H₃PO₄, aldehyde acids such as hydroxy alkyl phosphonic acids, such as hydroxyl butyl phosphonic acid and hydroxyl pentyl phosphonic acid, and the like, from the hydroformylation system allows one to control the acidity of the hydroformylation reaction medium, thereby stabilizing the useful organophosphorous ligand by preventing or lessening its hydrolytic decomposition. Without being bound by theory, in a preferred embodiment, it is thought that adding the water-soluble amine to the process and allowing it to flow throughout the process enables it to neutralize the acids as they are formed. Since the water-soluble amine is available early in the process, much lower levels of amine are needed compared to the prior art, yet surprisingly very effective pH control and, thus, activity and ligand decomposition performance, are observed without detectable increases in heavies formation. If the amine is added to the extraction zone, then acid neutralization still occurs via migration of some of the amine into the organic phase and/or acid migration into the alkaline aqueous phase and the overall partition greatly favors the removal of the acidic species (either as free acid or neutralizes salt) into the aqueous phase. The use of oxy-acid salt solutions is restricted to the extraction zone since these salts or derivatives thereof have sparing solubility in the organic reaction fluid thus could cause fouling issues such as described in U.S. Pat. No. 8,884,072.

In one embodiment of the invention, epoxide additives can be employed to mitigate strongly acidic impurities as taught in U.S. Pat. No. 9,328,047. The epoxide additives may be added continuously or on an “as needed” basis. The resulting epoxide adduct will also be removed by the extractor and this removal is enhanced by the presence of low levels of water-soluble amines. The preferred epoxides are water-soluble or slightly water-soluble (their solubility being increased when they react with the acidic species) such that the adducts are efficiently removed from the system, e.g. via the extraction zone aqueous effluent stream.

The hydroformylation process may be conducted in any batch, continuous or semi-continuous fashion and may involve any catalyst liquid and/or gas recycle operation desired. The particular hydroformylation process for producing aldehydes from an olefinic unsaturated compound, as well as the reaction conditions and ingredients of the hydroformylation process are not critical features of this invention.

In a preferred embodiment, the hydroformylation reaction fluid includes any fluid derived from any corresponding hydroformylation process that contains at least some amount of four different main ingredients or components, i.e., the aldehyde product, a metal-organophosphorous ligand complex catalyst, free organophosphorous ligand and a solvent for said catalyst and said free ligand. The hydroformylation reaction mixture compositions can and normally will contain additional ingredients such as those that have either been deliberately employed in the hydroformylation process or formed in situ during said process. Examples of such additional ingredients include unreacted olefin starting material, carbon monoxide and hydrogen gases, inert impurities that enter the system with the feeds, such as methane, carbon dioxide, and the like, and in situ formed by-products, such as saturated hydrocarbons and/or unreacted isomerized olefins corresponding to the olefin starting materials, ligand degradation compounds, and high boiling liquid aldehyde condensation by-products, as well as other inert co-solvent type materials or hydrocarbon additives, if employed.

The reaction conditions of the hydroformylation process may include any suitable hydroformylation conditions heretofore employed for producing optically active and/or non-optically active aldehydes. The hydroformylation reaction conditions employed will be governed by the type of aldehyde product desired as is generally known in the art.

The hydroformylation process may be carried out using one or more suitable reactors such as, for example, a fixed bed reactor, a fluid bed reactor, a plug-flow reactor, a continuous stirred tank reactor (CSTR) or a slurry reactor. The optimum size and shape of the reactor will depend on the type of reactor used. The reaction zone employed may be a single vessel or may comprise two or more discrete vessels in series or in parallel. The reaction steps may be affected by the incremental addition of one of the starting materials to the other. Also, the reaction steps can be combined by the joint addition of the starting materials. When complete conversion is not desired or not obtainable, the starting materials can be separated from the product, for example by distillation, and the starting materials then recycled back into the reaction zone.

The extraction zone employed in this invention may be a single vessel or may comprise two or more discreet vessels. In one embodiment of the invention, a reaction vessel may be employed as an extractor, e.g., when the process is operated in batch mode.

The recycle procedure generally involves withdrawing a portion of the liquid reaction medium containing the catalyst and aldehyde product from the hydroformylation reactor, i.e., reaction zone, either continuously or intermittently, and recovering the aldehyde product therefrom by use of a composite membrane, such as disclosed in U.S. Pat. Nos. 5,430,194 and 5,681,473, or by the more conventional and preferred method of distilling it, i.e., vaporization separation, in one or more stages under normal, reduced or elevated pressure, as appropriate, in a separate distillation zone, the non-volatilized metal catalyst containing residue being recycled to the reaction zone as disclosed, for example, in U.S. Pat. No. 5,288,918. Condensation of the volatilized materials, and separation and further recovery thereof, e.g., by further distillation, can be carried out in any conventional manner, and the crude aldehyde product can be passed on for further purification and isomer separation, hydrogenation, oxidation, and/or condensation, if desired, and any recovered reactants, e.g., olefinic starting material and syngas, can be recycled in any desired manner to the hydroformylation zone (reactor). The recovered metal catalyst-containing raffinate of such membrane separation or recovered non-volatilized metal catalyst-containing residue of such vaporization separation can be recycled to the hydroformylation zone (reactor) in any conventional manner desired.

The materials of construction are not particularly critical to the invention and can readily be chosen by one of ordinary skill in the art. The hydroformylation process may be conducted in, for example, glass lined, stainless steel or similar type reaction equipment. The equipment used to recycle the water should be compatible with traces of butyric acid to avoid corrosion issues. The reaction zone may be fitted with one or more internal and/or external heat exchanger(s) in order to control undue temperature fluctuations, or to prevent any possible “runaway” reaction temperatures.

It is generally preferred to carry out the hydroformylation process in a continuous manner. Continuous hydroformylation processes are well known in the art, with or without olefin and/or catalyst recycle.

The separation zone employed may be a single vessel or may comprise two or more discrete vessels. In one embodiment, the aldehyde product mixtures may be separated from the other components of the crude reaction mixtures in which the aldehyde mixtures are produced by any suitable method such as, for example, solvent extraction, crystallization, distillation, vaporization, wiped film evaporation, falling film evaporation, phase separation, filtration, and the like or any combination thereof. The reaction zone(s) and separation zone(s) employed herein may exist in the same vessel or in different vessels. For example, reactive separation techniques such as reactive distillation, reactive membrane separation, and the like, may occur in the reaction zone(s). One method for separating the aldehyde mixtures from the other components of the crude reaction mixtures is by membrane separation, which is described, for example in U.S. Pat. Nos. 5,430,194 and 5,681,473.

More particularly, distillation and separation of the desired aldehyde product from the metal-organophosphorous complex catalyst containing reaction fluid may take place at any suitable temperature desired. In general, it is preferred that such distillation take place at relatively low temperatures, such as below 150° C., and more preferably at a temperature in the range of from 50° C. to 140° C. It is also generally preferred that such aldehyde distillation take place under reduced pressure, e.g., a total gas pressure that is substantially lower than the total gas pressure employed during hydroformylation when low boiling aldehydes (e.g., C₄ to C₆) are involved or under vacuum when high boiling aldehydes (e.g., C₇ or greater) are involved. For instance, a common practice is to subject the liquid reaction product medium removed from the hydroformylation reactor to a pressure reduction so as to volatilize a substantial portion of the unreacted gases dissolved in the liquid medium that now contains a much lower synthesis gas concentration than is present in the reaction medium to the distillation zone, e.g., vaporizer/separator, wherein the desired aldehyde product is distilled. In general, distillation pressures ranging from vacuum pressures on up to total gas pressure of 340 kPa should be sufficient for most purposes.

The conditions for aldehyde isomer separation and removal of a water phase are not narrowly restricted and are well known and disclosed, for example, in CN115417746 and CN217612992U. These patents do not reuse the water streams. In most cases, the crude mixed aldehyde is treated to remove dissolved gases such as syngas, N₂, and other non-condensable gases prior to the isomer separation process. This syngas removal process is usually done in a distillation system such as shown in CN115417746, for example.

The aldol reaction with aldehydes is a well-known process. This usually involves the self-condensation of the aldehyde to make the corresponding “dimer”. Cross aldolization is also known (two different aldehydes). At least one mole of water is generated which is removed in one of two places; in the overhead of the aldol vent system (typically in part as the water-aldehyde azeotrope present in the vent or the top of a reactive distillation system) or in the aqueous aldol catalyst purge. Typical conditions for this reaction are given in U.S. Pat. No. 5,434,313A. The redistillation of the aldol catalyst purge are the same as for the extractor tails and the preferred option is using a steam stripper such as described in U.S. Pat. No. 5,744,649.

The use of a distillation process such as a steam stripper to remove organics from an aqueous stream is well known and is taught in U.S. Pat. No. 5,744,649 except a portion of the decanted water phase is not returned to the stripper but used in the present invention. In many cases, several of the aqueous streams described above are fed to this stripper prior to the stripped aqueous phase being sent to the wastewater treatment facility which reduces the COD (chemical oxygen demand) or the BOD (biological oxygen demand). The conditions within the stripper are not narrowly restricted.

In one preferred embodiment, where multiple streams are available for the steam stripper, the use of the aldol catalyst purge and/or an extractor aqueous purge that used buffered water is preferred as these will control the pH of the distillation bottoms to mitigate any carboxylic acid that might be present in any of the streams. If the aldol catalyst purge is one of the streams being treated, the condensed overhead aqueous phase should be monitored for pH to insure minimal amounts of caustic are entrained during the distillation.

Illustrative non-optically active aldehyde products include e.g., propionaldehyde, n-butyraldehyde, isobutyraldehyde, n-valeraldehyde, 2-methyl 1-butyraldehyde, hexanal, hydroxyhexanal, 2-methyl 1-heptanal, nonanal, 2-methyl-1-octanal, decanal, adipaldehyde, 2-methylglutaraldehyde, 2-methyladipaldehyde, 3-hydroxypropionaldehyde, 6-hydroxyhexanal, alkenals, e.g., 2-, 3- and 4-pentenal, alkyl 5-formylvalerate, 2-methyl-1-nonanal, 2-methyl 1-decanal, 3-propyl-1-undecanal, pentadecanal, 3-propyl-1-hexadecanal, eicosanal, 2-methyl-1-tricosanal, pentacosanal, 2-methyl-1-tetracosanal, nonacosanal, 2-methyl-1-octacosanal, hentriacontanal, 2-methyl-1-triacontanal, and the like.

Illustrative optically active aldehyde products include (enantiomeric) aldehyde compounds prepared by the asymmetric hydroformylation process of this invention such as, e.g., S-2-(p-isobutylphenyl)-propionaldehyde, S-2-(6-methoxy-2-naphthyl) propionaldehyde, S-2-(3-benzoylphenyl)-propionaldehyde, S-2-(3-fluoro-4-phenyl)phenylpropionaldehyde, and S-2-(2-methylacetaldehyde)-5-benzoylthiophene.

### SPECIFIC EMBODIMENTS OF THE INVENTION

All parts and percentages in the following examples are by weight unless otherwise indicated. Pressures are given as absolute pressure unless otherwise indicated. Compositions of solutions were determined by gas chromatography.

### Example 1

The composition of the water phase from the reactive distillation system for a butyraldehyde aldol unit such as described by U.S. Pat. No. 5,434,313 was found to have 96% water with 3 wt % butyraldehyde and traces of butanols in the feed to the steam stripper and the resulting stripped water phase was over 99.5 wt % water. In this case, the feed stream had been distilled, condensed, decanted, and the water layer sent to second steam stripper as shown in FIG. 1 of U.S. Pat. No. 5,434,313. The feed to the stripper or the resulting organic-depleted aqueous streams are both suitable for the present invention.

### Example 2

A crude mixed butyraldehyde from which residual syngas and other non-condensible gases had been removed was subjected to a conventional distillation process similar to that described in CN217612992U to separate the branched aldehyde (isobutyraldehyde) from normal butyraldehyde. The overhead condensed stream was sent to a phase separation device (operated at 50° C.) wherein the organic phase comprising mostly of isobutyraldehyde with water is recycled back to the distillation apparatus. The aqueous phase suitable for the present invention was found to be 95 wt % water and 5 wt % isobutyraldehyde.

### Example 3

A steam stripper to remove organic materials from a variety of propylene hydroformylation streams described herein was operated wherein the steam stripper removed an overhead stream that was cooled, and a condensed liquid phase was obtained. The feed streams included an aqueous extractor tails stream, the butyraldehyde isomer column aqueous phase similar to that of Example 2, and a small flow of a mostly water stream from butanol refining. The vaporized material was condensed, and the liquid phase was allowed to separate into an organic phase and an aqueous phase. The organic phase was removed and the aqueous phase was returned to the stripper analogous to the process described in U.S. Pat. No. 5,744,649. While the feed composition may vary, a typical composition of the decanted water phase was greater than 93 wt % water with 6 wt % of mixed aldehydes and butanols which would be suitable for the present invention. The organic phase consisted of butyraldehyde, butanols, and other organic materials and was processed elsewhere.

1. A process comprising: (1) conducting in a reaction zone a hydroformylation reaction employing a reaction fluid comprising (a) at least one acidic compound selected from a phosphorus acidic compound or carboxylic acid compound, (b) a metal-organophosphorus ligand complex catalyst that comprises a metal of Group 8, 9 or 10 complexed with an organophosphorous ligand, and, optionally, (c) free organophosphorus ligand; (2) contacting at least a portion of the reaction fluid with an aqueous extraction fluid to remove at least a portion of the acidic impurities; (3) at least partially separating in at least one extraction zone at least some of acidic compounds from the reaction fluid; and (4) removing the acidic compound from the at least one extraction zone via an extraction zone aqueous effluent stream;
wherein at least a portion of the aqueous extraction fluid used in step (2) comprises recycled water from the hydroformylation system or a subsequent processing step in an aldehyde production process wherein the water stream to be recycled contains the product aldehyde and this water stream is subjected to at least one distillation process followed by at least one water/aldehyde phase separation process to produce the aqueous extraction fluid.

2. The process of claim 1 wherein the aqueous extraction fluid further comprises an acid neutralizing agent to facilitate the removal of the phosphoric acid impurities.

3. The process of claim 2 wherein the acid neutralizing agent is added to the aqueous extraction fluid as an aqueous solution and where such aqueous solution was prepared using at least some of the recycled water.

4. The process of claim 2 wherein after step (4) a water-washing process is employed to further reduce water-soluble impurities wherein at least some of this wash water was derived from recycled water.

5. The process of claim 1, wherein the recycled water was recovered from the crude aldehyde mixture following the product/catalyst separation process such as a syngas removal column or an aldehyde isomer separation distillation process.

6. The process of claim 1, wherein the recycled water was recovered from an aldol reactive distillation system overhead stream.

7. The process of claim 1, wherein the recycled water was recovered from an aqueous extractor tails fluid that was subjected to a distillation process wherein the vaporized water is condensed and phase separated into an aqueous phase that is recycled in step (2).

8. The process of claim 1 wherein two or more sources of process water are combined and distilled to generate a mixture of water vapor and aldehyde vapor that is then cooled, condensed, and phase separated to yield an organic phase and a water phase and wherein at least a portion of the water phase is used as the recycled water.

9. The process of claim 2 wherein the acid neutralization agent comprises at least one of triethanolamine, methyldiethanolamine, ethyldiethanolamine, tri (2-hydroxypropyl)amine, or ethoxylates of any of these.

10. The process of claim 2 wherein the acid neutralization agent comprises at least one anion selected from the group consisting of phosphate, carbonate, citrate, maleate, and borate compounds and at least one cation selected from the group consisting of ammonium and alkali metals and mixtures thereof.

11. The process of claim 1, wherein the organophosphorous ligand is a hydrolyzable organophosphorous ligand.

12. The process of claim 1, wherein the initial water generated by a distillation process followed by a phase separation process is subjected to a second distillation step prior to being recycled.

13. In a hydroformylation process comprising the steps of: (1) conducting in a reaction zone a hydroformylation reaction employing a reaction fluid comprising (a) a phosphorus acidic compound, (b) a metal-organophosphorus ligand complex catalyst that comprises a metal of Group 8, 9 or 10 complexed with an organophosphorous ligand, and, optionally, (c) free organophosphorus ligand; (2) contacting at least a portion of the reaction fluid with a aqueous extraction fluid to remove at least a portion of the phosphorus acidic impurities; (3) at least partially separating in at least one extraction zone at least some of phosphorus acidic compounds from the reaction fluid; and (4) removing the phosphorus acidic compound from the at least one extraction zone via an extraction zone aqueous effluent stream; the improvement comprising: using recycled water from the hydroformylation system or a subsequent processing step in an aldehyde production process wherein the water stream to be recycled contains the product aldehyde which has been subjected to at least one distillation process followed by at least one water/aldehyde phase separation process to produce the aqueous extraction fluid to form at least a portion of the aqueous extraction fluid in step (2).
